(19) United States
(12) Patent Application Publication
(10) Pub. No.: US 2014/0377228 A1
(43) Pub. Date: Dec. 25, 2014

(54) Bone Marrow-Derived Cells Ameliorates The Pathological Consequences Of The Liver In Case Of Alpha1-Antitrypsin Deficiency

(71) Applicant: NATIONAL INSTITUTE OF IMMUNOLOGY, New Dellhi (IN)

(72) Inventors: Prakash Baliger, New Delhi (IN); Jeffrey Teckman, SAINT LOUIS, MO (US); Asok Mukhopadhyay, New Delhi (IN)

(73) Assignee: NATIONAL INSTITUTE OF IMMUNOLOGY, New Dellhi (IN)

(21) Appl. No.: 14/109,212

(22) Filed: Dec. 17, 2013

Related U.S. Application Data
(63) Continuation-in-part of application No. 12/915,214, filed on Oct. 29, 2010.
(60) Provisional application No. 61/280,188, filed on Oct. 30, 2009.

(51) Int. Cl. A61K 35/28

(52) U.S. Cl. CPC A61K 35/28; A61K 2035/124
USPC 424/93.7

(57) ABSTRACT

The present invention is based on the findings that bone marrow (BM)-derived progenitor cells more specifically mesenchymal stem cells (MSCs), hematopoietic stem cells (HSCs) and uncommitted hematopoietic cells (lin⁻) are capable of regenerating liver in case of injury. The invention provides a method for treating genetic disorder like Alpha1-antitrypsin deficiency (A1-ATD) by administering BM derived Lin⁻ cells in human mutant A1-AT expressing transgenic mouse model. The invention also provides the state of art for replacement of mutant host hepatocytes by transplanting wild-type uncommitted donor (lin⁻) cells.

### CROSS REFERENCE TO RELATED APPLICATIONS

This application is a continuation-in-part application of U.S. application Ser. No. 12/915,214 filed Oct. 29, 2010, which claims the benefit of U.S. Provisional Patent Application 61/280,188 filed Oct. 30, 2009 incorporated by reference.

### BACKGROUND OF THE INVENTION

1. Field of the Invention

This invention is in the field of stem cell therapy, more particularly in the use of BM-derived lin⁻ cells to treat Alpha 1-antitrypsin deficiency (A1-ATD) by replacing the affected hepatocytes. More particularly, the invention relates to a method of treating A1-ATD by use of lin⁻ BM cells.

2. Technical Background

In the past decades, it has been shown that BM-derived stem cells are capable of regenerating liver in the event of any injury to the organ. A1-AT is glycoprotein in nature, a protease inhibitor belonging to the serpin superfamily, and secreted primarily by hepatocytes, inhibits neutrophil elastase, which is a protease that degrades connective tissue of the lung. If this protein does not properly fold (in case of disease, A1-ATD) due to some changes (mutation) in the encoding gene, the protein aggregated inside ER causing stress leading to death of hepatocytes and liver damage. Worldwide, about 3.4 million people are affected by the disease with different allele combinations (PiSS, PiSZ and PiZZ), which may cause damage to the liver, or the lung, or both (De Serres F J, 2002). A1-ATD arises from the homozygous inheritance of the A1-AT-Z allele (A1-AT-ZZ), a variant of SERPINA1/A1-AT resulting from substitution of lysine for glutamate at 342 (Lomas et al., 1992).

The physiological function of A1-AT is the inhibition of neutrophil elastase, cathepsin G and proteinase 3. The major site of A1-AT synthesis is liver; however, a small amount of this protease inhibitor is also synthesized by the extra-hepatic tissues/cells, including macrophages, intestinal epithelial cells and intestinal Paneth cells. In the liver, the secretory glycoprotein undergoes a series of transient interactions with molecular chaperones in the endoplasmic reticulum (ER) until the folding or assembling process is completed. If a translocation-competent conformation of protein is formed, the secretory glycoprotein molecule dissociates from molecular chaperones allowing subsequent transport; otherwise, it forms abnormally folded PiZZ A1-AT molecule which do not dissociate from their chaperones and thus are retained in the ER.

The major pathological change in A1-ATD is the formation of ‘periodic acid Schiff’ positive diastase-resistant globules in the ER of hepatocytes. It has been evident in transgenic mice that the liver injury in case of A1-ATD is directly related with the retention of the abnormally folded A1-AT protein in the ER of the liver (Dycaico et al., 1988; Carlson et al., 1989). Pathological changes may also occur in the connective tissue of the lung due to uncontrolled protease activity, which causes the development of end stage pulmonary emphysema. A1-AT has been found to constitute greater than 90% of the neutrophil elastase inhibitor activity in pulmonary alveolar lavage fluid.

The current available treatments of genetic liver diseases are limited. Liver transplantation (LT) has been used for the treatment of genetic liver diseases, such as urea cycle defect, LDL receptor deficiency, HT1, A1-ATD, etc. (Whitington et al., 1988; Kaylor et al., 2003). Currently, split liver transplantation is used to supply a part of normal gene function in case of genetic liver diseases rather than complete replacement of liver. In case of A1-ATD, some patients develop end-stage liver disease because the mutant protein accumulates in the liver; other variants of the mutations have a little or no liver disease but patients develop end-stage pulmonary emphysema. Affected individuals with severe lung disease might require both lung and liver transplantation. A study in an animal model showed that primary hepatocytes transplantation leads to repopulation of normal hepatocytes with the reduction of mutated human A1-AT protein in the transgenic mice (Dine et al., 2011).

Hepatocyte transplantation has been used as an alternative to LT in patients with congenital metabolic disorders (Fox et al., 1998). A1-ATD and ornithine transcarbamoylase deficiency (Strom et al., 1997), and other genetic liver diseases. The limiting factor for LT or hepatocyte transplantation is the availability of donor liver and immune-rejection. Repeated transplantation may be required to maintain the therapeutic effects. The problem is augmented due to a shortage of donor livers.

U.S. Pat. No. 6,403,646B1 refers to a method for treatment of A1-ATD using protease inhibitor of type Z mutation by administration of phenylbutyric acid derivatives, and also discloses methods for the prevention and treatment of liver injury and emphysema associated with A1-ATD.

U.S. Pat. No. 6,656,912B2 refers to treatment of therapeutic or prophylactic level; conditions associated with A1-ATD in a subject with an effective amount of a glycosidase inhibitor or an imino-sugar or reduced imino-sugar. The glucosidase inhibitor is castanospermine.

### SUMMARY OF THE INVENTION

This invention pertains to the field of stem cell therapy in genetic liver disease, A1-ATD (due to PiSS, PiSZ and PiZZ allele combinations). Bone Marrow (BM)-derived uncommitted cells having the capacity of differentiating into different liver types are disclosed. These uncommitted stem cells are capable of differentiating into hepatocyte-, endothelial- and Kupffer-like cells.

An aspect of the invention is the use of BM-derived uncommitted cells for restoring functional A1-AT synthesis in a mammal.

Another aspect of the invention is the use of stem cells, regenerative cells and angiogenesis promoting cells for restoring functional A1-AT synthesis in a mammal. More specifically, the invention relates to the use of Bone-Marrow-derived cells and specialized progenitor cells and products thereof.

Still another aspect of the invention is a method and compositions for treating or preventing consequences of A1-ATD in a mammal.

Yet another aspect of the invention is administering a therapeutically effective number of cells differ cells that are capable of inducing regenerating of the liver and restoring its normal function.

### BRIEF DESCRIPTION OF FIGURES

FIGS. 1A-1G: Diastase treated PAS staining of wild type control C57B16/J mice (FIG. 1A), age matched experimental control PiZZ mouse (FIG. 1B, 1D, 1F) and after 1 month (FIG. 1C), 3 months (FIG. 1E) and 6 months (FIG. 1G) of transplantation of lin⁻ cells. Representative images are shown (magnification 200×).

FIG. 2: Quantitative analysis of the globules-containing and globules-free hepatocytes in wild type mouse, age matched experimental control PiZZ mouse (black color bar) and after 1, 3 and 6 months of transplantation (gray color bar) of lin⁻ cells. Analysis was done on the basis of counting globules-containing and globules-free hepatocytes in 30 fields (FIG. 1, 200×) from multiple liver sections of 2 mice (each type).

FIGS. 3A-3C: The presence of GFP⁺ donor cells in PiZZ mouse liver after 1 month (FIG. 3A, first row), 3 months (FIG. 3B, second row) and 6 months (FIG. 3C, third row) of transplantation of lin⁻ cells. Representative images are shown (magnification 200×).

FIG. 4: Quantitative analysis of donor-derived cells in PiZZ mouse liver after 1, 3 and 6 months of transplantation of lin⁻ cells. Analysis was done on the basis of counting total nuclei in GFP⁺ and GFP⁻ cells in 20 fields (FIG. 3, 200×) from multiple liver sections of 2 mice (each type).

FIGS. 5A-5C: Immunohistochemical staining of liver section for co-expression of GFP and Albumin in PiZZ mouse liver after 1 month (FIG. 5A, first row), 3 months (FIG. 5B, second row) and 6 months (FIG. 5C, third row) of transplantation of lin⁻ cells. Representative images are shown (magnification 200×).

FIG. 6: Quantitative analysis of donor-derived hepatocyte-like cells expressing both GFP and albumin in PiZZ mouse liver after 1, 3 and 6 months of transplantation of lin⁻ cells. Analysis was done on the basis of counting total GFP⁺Alb⁺ and GFP⁻Alb⁺ cells in 20 fields (FIG. 5, 200×) from multiple liver sections of 2 mice (each type).

FIGS. 7A-7G: Collagen deposition by picrosirus red staining of wild type control C57B16/J mice (FIG. 7A), age matched experimental control PiZZ mouse (FIG. 7B, 7D, 7F) and after 1 month (FIG. 7C), 3 months (FIG. 7E) and 6 months (FIG. 7G) of transplantation of lin⁻ cells. Representative images are shown (magnification 100×).

FIG. 8: Collagen proportionate area (CPA) in normal control C57B16/J mouse, age matched experimental control PiZZ mouse and after 1, 3 and 6 months of transplantation of lin⁻ cells. Analysis was done on the basis of determining area of stained zone with respect to the total area per field using Image J software in 30 fields (FIG. 7, 100×) from multiple liver sections of 2 mice (each type).

FIGS. 9A and 9B: Glycogen levels in liver tissue (FIG. 9A) and glucose levels (FIG. 9B) in serum of normal control C57B16/J mouse, control PiZZ mouse and after transplantation of lin⁻ cells. Analysis was done by enzymatic method. The glucose obtained from the liver tissue and serum samples were determined by GOD-POD method according to protocol provided by manufacturer of the kit (ERBA Mannheim-Transasia, India).

FIG. 10: Inflammatory response in PiZZ and transplanted mice. Bar diagram showing comparative inflammatory score of liver tissue sections of age matched experimental sham control PiZZ mice and after 1, 3, 6 and 12 months of transplantation of Lin⁻ BM cells. The results showing no significant effect of transplantation on inflammatory response up to 6 months, thereafter the same was significantly suppressed.

### DEFINITIONS

BM cells: Bone marrow is the site where in adults all blood cells are developed from a population of ancestor cells, which reside in the marrow niche, termed as hematopoietic stem cells (HSCs). HSCs differentiate into uncommitted progenitor cells, which later committed into various blood cells, broadly classified into lymphoid (T and B cells) and myeloid lineage (monocytes, neutrophils, macrophages, megakaryocytes, platelets, erthrocytes, cosinophilia, mast cells). Besides HSCs, other kinds of stem cells are present in the bone marrow, which primarily support hematopoietic cells for their maintenance, proliferation and differentiation, known as mesenchymal stem cells (MSCs). Adipocytes, chondrocytes, osteoblasts and fibroblasts originate from MSCs. A third category of cells that are present in the bone marrow is known as endothelial progenitor cells (EPCs). Thus, bone marrow is highly heterogeneous population of cells with diverse biological functions. Lin⁻ BM cells; These are partially purified BM cells, sometimes referred to as uncommitted BM cells. Crude BM cells, depleted for CD5, CD11b, CD45R, 7-4, Gr-1, and Ter119-Expressing cells, are termed as Lin⁻ BM cells. These cells are also heterogeneous in nature, consisting of mainly HSCs and their uncommitted progeny (progenitor cells), MSCs and their lineage cells as described above, and EPCs. Upon magnetic depletion of committed cells the majority (>90%) of the negatively selected cells are found to be hematopoietic origin (HSCs and uncommitted hematopoietic progenitor cells), and the remaining <10% cells consist of MSCs and other cells.

Competent or therapeutically active cells: A small fraction of cells belonging to Lin⁻ category, which have the potential to differentiate into hepatocytes and synthesize target protein (A1-AT) are termed as competent or therapeutically active cells.

### DETAILED DESCRIPTION OF THE INVENTION

This invention pertains to the field of stem cell therapy in genetic liver disease, A1-ATD (due to PiSS, PiSZ and PiZZ allele combinations). More particularly it relates to cells useful for restoring A1-AT synthesis, including stem cells, regenerative cells and angiogenesis promoting cells. More specifically, the invention relates to the use of bone marrow derived cells and specialized progenitor cells and products thereof.

Accordingly, provided herein are methods and compositions for treating on preventing consequences of A1-ATD in a mammal, particularly a humans.

In another aspect, a therapeutically effective amount of cells are administered that are capable of inducing regeneration of the liver and restore its normal function. In one aspect a therapeutically effective amount of cells are administered that are capable of inhibiting hepatocyte degeneration and hepatic fibrosis/cirrhosis. Another aspect of the invention may be the prevention of hepatocellular carcinoma and emphysema of the recipient resulting from A1-ATD.

The invention teaches the use of adult stem and progenitor cells for the treatment of A1-ATD. Specific properties of stem cells that are suitable for use in practicing the current invention are ability to engraft in the damaged tissue, ability to perform normal function, ability to prevent atrophy, as well as to differentiate into hepatocytes, endothelial cells and other cells. In the case of human patients with A1-ATD (Carriers of PiZZ allele) stem cells are collected from allogenic sources, expanded ex vivo or kept unmanipulated, and introduced to the patient at a concentration and frequency to result in therapeutic benefit. The stem cells are selected for the ability to cause: their engraftment, prevention of atrophy and regeneration of the liver parenchymal and mesenchymal cells. Stem cells are chosen from the human bone marrow, specifically MSCs, and HSCs, uncommitted human BM progenitor cells, cells from human umbilical cord blood or Wharton jelly. Bone marrow umbilical cord and Wharton jelly-derived MSCs have the ability to differentiate into helpatocytes and endothelial cells that can involve in the liver regeneration (Kim et al., 2013). Besides these, MSCs have immunoregulatory and immunomodulatory effects on the recipient's immune cells (Ghannam et al., 2010; Shi et al., 2011).

The competent cells of BM engraft and differentiate into hepatocytes. In diseased liver, the hepatocytes, harboring mutated A1-AT protein is aggregated. As the cells cannot retain this miss-folded protein for linger duration, they die. Due to compensatory mechanism, other host hepatocytes (not affected) along with BM-derived hepatocytes proliferate and replace some dead hepatocytes. This process of self-destruction of host hepatocytes and proliferation of BM-derived hepatocytes, which do not harbor any mutated A1-AT, continues. As a result, with time, the majority of the affected host hepatocytes are replaced with neo-hepatocyctes-derived from BM.

The transplanted cells are involved in regeneration of hepatic parenchymal and non-parenchymal cells.

### BM Derived Stem Cells and Selection of Stem Cells for the Treatment of A1-ATD

MSCs are characterized by the expression of cell surface markers such as CD45⁻CD73⁺CD29⁺CD90⁺CD105⁺ whereas HSCs are characterize by lin⁻c-kit⁺Sca-1⁺ (LSK). The lin⁻ cells are characterized primarily by a mixture of uncommitted HSCs and cells of the MSC lineage. The adult stem cell-based therapy has been proposed as an attractive and novel approach to treat degenerative or genetic liver diseases such as HT-1 (Lagasse et al., 2001), Crigler-Naijar syndrome (Muraca et al., 2007), Familial progressive cholestasis type 2 (Chen et al., 2008), Hemophila A (Yadav et al., 2009). Because of their definite self-renewal capacity and differentiation potential, adult stem cells are considered ideal vehicles for permanent delivery of normal proteins/factors to those tissues affected by loss-of-function due to genetic mutations. For example, uncommitted hematopoietic cells are transplanted into factor VIII deficient mouse, which give rise to hepatocytes and endothelial cells, and synthesize functional factor VIII protein (Yadav et al., 2009).

BM cells were isolated in a two stage process from 6- to 8-week eGFP transgenic C57B16/J mice. In the first step, cells were removed from tibia and femurs by flushing with medium. The firmly adhered cells of the endosteal region were recovered in the second stage by digesting the crushed bone with collagenase type IV (0.03%) and dispase enzyme (2U/ml). BM cells were pooled in a tube and erythrocytes were lysed by treatment with Gey's solution (Sushmita et al., 2013). After washing the cells with media, cells were plated on a 90 mm culture-disc with 10% FCS-DMEM culture medium and incubated at 37° C. in 5% CO₂ and 95% O₂. To obtain bone marrow MSCs, after 24 hrs of the culture, nonadhered and dead cells were removed by washing 2-3 times. The MSC markers (positive for CD73, CD90, CD105 and negative for CD45, CD14, CD34) were studied in Passage 1 or 2. To obtain HSCs, the isolated BM cells were sorted on the bases of phenotypic markers (LSK). To obtain Lin⁻ cells, the isolated BM cells were subjected to Magnetic Assisted Cells Sorting (MACS) by linage negative selection kit.

Human umbilical cords were collected in Hank's balanced salt solution (HBSS) at 4° C. After disinfection with 70% alcohol, core vessels were cleared off. The mesenchymal tissue (in Wharton's jelly) was cut into cubes (approx. 0.5 cm³) and centrifuged 250 g for 5 min. The tissue pellet was washed with serum free DMEM. The tissue pellet (mesenchymal tissue) was digested with collagenase at 37√ C. for 18 hrs, washed and further digested with 0.25% trypsin for 30 min at 37° C. FBS were used to neutralize the excess trypsin. Then the suspension was washed with DMEM and cultured in 10% FCS-DMEM. These cultured cells were confirmed by their phenotypic expression of MSCs markers. In alternate, the small pieces of cord tissue are cultured (explant culture) in above medium, the MSCs are migrated to the culture flask and make fibroblastic colonies.

Applicants intend to study human cord blood and tissue derived HSCs and MSCs, respectively for the treatment of alpha1antitrypsin deficient in PiZZ-SCID mouse. hMSCs/HSCs could home to the sites of damage liver, and differentiate into hepatocyte, and become functionally active like normal hepatocyte and start synthesizing human A1-AT. MSCs are known to constitutively secrete extracellular matrix-degrading enzymes, primarily matrix metalloproteinase 9, which may help in degrading fibrous tissue in the liver as the diseased mouse may cause fibrosis/cirrhosis. In addition, it has been reported that MSCs also secrete several growth factors including VEGF, PDGF, and other hematopoietic growth factors (Beckermann et al., 2008).

### Method of Administration

In the method described herein, the therapeutically effective number of the BM derived stem/progenitor cells such as MSCs, HSCs and lin⁻ cells are transplanted in the mutant human A1-AT (ZZ variant)-expressing mouse through the intrasplenic route. Generally, the range of therapeutically effective doses of the cells is between about 0.1×10⁶ to about 20×10⁶ Lin⁻ BM cells. The number of cells that are administered at one time as a single dose is about range 0.25×10⁶ Lin⁻ cells. This is based on the weight of the liver. The cells are administered intrasplenically as described by Stock et al., (2010). It is expected that for humans and other large mammals the dose would be approximately 5 million to 20 million Lin⁻ BM cells per 100 grams of liver.

In humans and in larger mammals, cells can be transplanted through routes like (a) peripheral vein/IV, (b) intraplenic, (c) portal vein, and (d) hepatic artery. Cells may be suspended in normal saline.

Except as defined herein, all the technical and scientific terms used herein have the same meaning as commonly understood by one of ordinary skill in the art to which the invention belongs. The terms “a”, “an” and “the” refers to “one or more” when used in the subject specification, including the claims. Thus, for example, reference to “a compound” may include a plurality of such compounds.

It should also be noted that the term “or” is generally employed in its sense including “and/or” unless the content clearly dictates otherwise.

The following abbreviations or terms are used herein:
A1AT—alpha 1-antitrypsin
A1—ATD-Alpha 1-antitrypsin deficiency
ALT—alanine aminotransferase
BM—bone marrow
BMC—bone marrow cell
DMEM—Dulbecco's Modified Eagle Medium
ER—Endoplasmic reticulum
FCS—Fetal calf serum
HBSS—Hank's balanced salt solution
HGF—hepatocyte growth factor
HSC—Hematopoietic stem cells
1HC—immunohistochemistry
IVC—individual ventilated cages
LSEC—liver sinusoidal endothelial cell
LSK—lin⁻e-kit⁺Sca-1⁺
LT—Liver Transplantation
MACS—Magnetic assisted cell sorting

It should be understood that the detailed description and specific examples, while indicating aspects of the invention, are given by way of illustration only, since various changes and modifications within the spirit and scope of the invention will become apparent to those skilled m the art. One skilled in the art, based upon the description herein, may utilize the present invention to its fullest extent. The invention will be further explained by the following illustrative examples that are intended to be non-limiting, are to be construed as merely illustrative, and not limitative of the remainder of the disclosure in any way whatsoever.

Other systems, methods, features and advantages of the present invention are or will be apparent to one of skill in the art.

### EXAMPLES

### Animals

Transgenic mice of C57B16/J background, expressing human mutant A1-AT, were obtained from Prof Jeffery Teckman of Saint Louis University, Mo., USA (Rudnick et al., 2002). Mice were maintained in the Experimental Animal Facility of the National Institute of Immunology (NII), New Delhi, India. Mice were maintained in IVC on 12-hour dark-light cycles and on ad libitum mouse chow and water. All experiments were approved by the Animal Ethics Committee of NII.

### Example 1

### Isolation and Purification of the Lin⁻ Cells

Lin⁻ (CD5, CD11b, CD45R, 7-4, Gr-1, and Ter119-depleted) BMCs were isolated from C57B16/J background eGFP transgenic female mice by magnetic cell sorter (Miltenyi Biotec., Germany) following a negative selection method. These mice express normal A1-AT, A total of 250,000 sorted cells were transplanted intrasplenically in PiZZ mice. Saline injected PiZZ mice were used as control throughout the invention.

### Example 2

### Animal Surgery

All animal procedures were approved by the Institutional Animal Ethics committee (IAEC) of the National Institute of Immunology. Animals were anesthetized with intraperitioneal injection of Ketamin (100 mg/kg b wt) and xylazine (19 mg/kg b wt). An area of 2 sq. cm underneath the most caudal rib was shaved and was disinfected with iodine and alcohol. A pre-arranged ligation per animal using at least 25 cm of the surgical suture with knot was prepared. The abdominal cavity was carefully opened with small scissors and the skin lifted with straight forceps to locate the spleen. Forceps were used to lift the spleen by grasping the adipose tissue adherent to the spleen. A pre-arranged ligation was placed over the spleen and the wooden end of a cotton applicator was pressed between the adherent adipose tissue and the spleen to expose the organ during the injection of the cells. The ligation was wound around the spleen, the end of the suture was held and the spleen was lifted. The pole of the spleen was punctured and the needle was pushed behind the ligation. The ligation was set tighter and the cells were slowly infused over a period of 1 to 2 min until the suspension is completely injected. Sham-operated animals were infused with saline instead of cells. The spleen was placed into the abdominal cavity. The abdominal cavity was closed by a normal running suture for the abdominal musculature and the skin. The animals were rehydrated by subcutaneous injection of 5 ml of sterile saline solution per 100 g body weight. Postoperative analgesia were omitted to avoid potential fulminant liver failure. All the animals were transferred into a pre-warmed cage immediately after surgery and allowed free access to normal drinking water and food.

### Example 3

### Presence of Donor Cells in the Recipient Mice

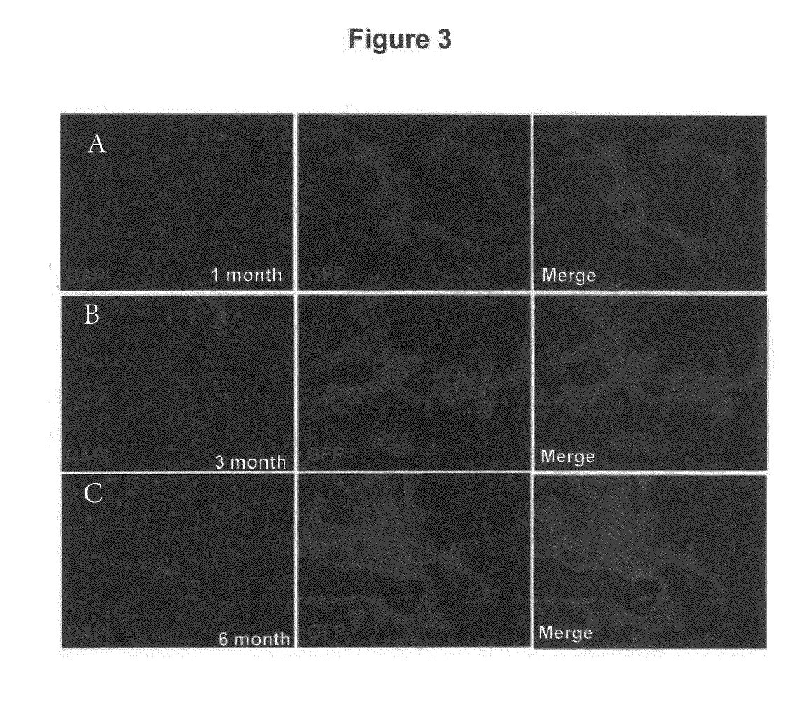

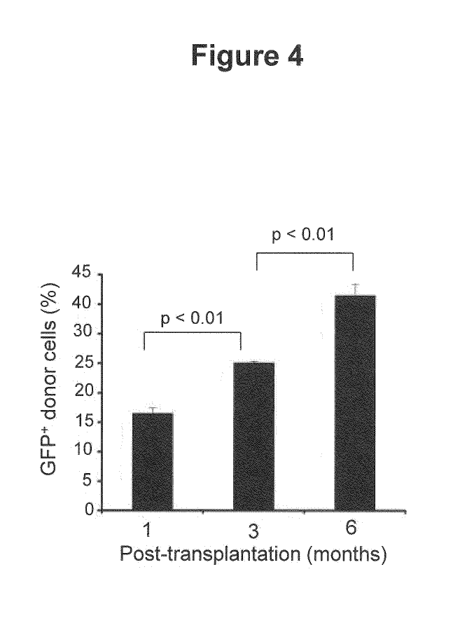

Immunohistochemical identification of the donor derived cells i.e GFP cells were quantitatively determined by examining the IHC sections. Cryosections of 5 μm were prepared from the recipient mice 1, 3 and 6 months after transplantation. Tissue were fixed with 4% paraformaldehyde in phosphate buffered saline and kept overnight in 30% sucrose solution before sectioning. Sections were first blocked with 1% BSA for 30 min at room temperature and permeabilized with 0.1% Titron X-100 for 30 min at room temperature. The sections were washed with PBS and incubated with primary anti-GFP antisera overnight at 4° C. The antisera were rinsed-off with PBS and the sections were incubated with the respective secondary anti-sera for 1-2 hours at room temperature. Sections were then washed with PBS and DAPI was added for nuclear staining, incubated for 5-10 min. Again slides were washed once with PBS and sections were covered using a cover slip containing fluorescent mounting medium. Fluorescent images were obtained using Olympus fluorescent microscope equipped with DP camera. Imaging (200× magnification) Imaging was done randomly about twenty fields of multiple sections avoiding the overlapping fields. The GFP stained cells were counted by using cell counter of the ImageJ (NIH) software (FIG. 3 and FIG. 4).

### Example 4

### Co-Expression of GFP and Albumin in Transplanted Cells

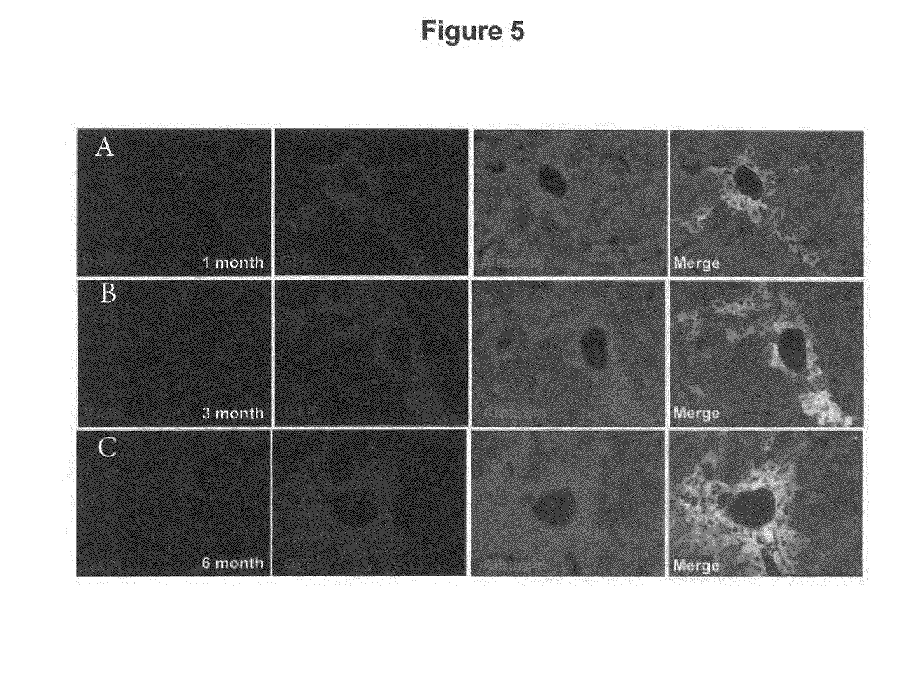

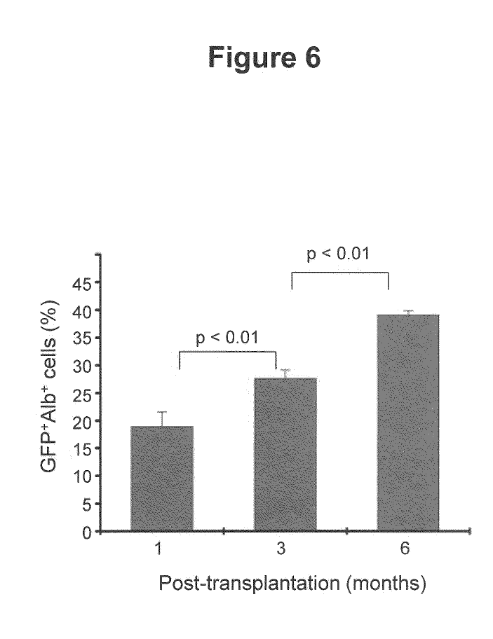

Immunohistochemical identification of the donor derived hepatocytes was analyzed. To know the functional status of the donor derived GFP cells, albumin and GFP co-staining was done. Cryosections of 5 μm were prepared from the recipient mice at 1, 3 and 6 month after transplantation. Tissue were fixed with 4% paraformaldehyde in phosphate buffered saline and kept overnight in 30% sucrose solution before sectioning. Sections were first blocked with 1% BSA for 30 min at room temperature and permeabilized with 0.1% Titron X-100 for 30 min at room temperature. The sections were washed with PBS and incubated with primary antibodies (anti-GFP and anti-albumin) for overnight at 4° C. The primary antibodies were rinsed-off with PBS and the sections were incubated with the respective secondary antibodies for 1-2 hours at room temperature. Sections were then washed with PBS and DAPI was added for nuclear staining, and further incubated for 5-10 min. Again slides were washed once with PBS and sections were covered with a cover slip containing fluorescent mounting medium. Fluorescent images were obtained using Olympus fluorescent microscope equipped with DP camera. Imaging was done randomly at 200× magnification in about twenty fields of multiple sections avoiding the overlapping of fields. The GFP and albumin co-stained cells were counted by using cell counter of the ImageJ software (FIG. 5 and FIG. 6).

### Example 5

### Quantification of Diastase Resistant-PAS Positive Globules Containing Hepatocytes in the Transplanted PiZZ Mice

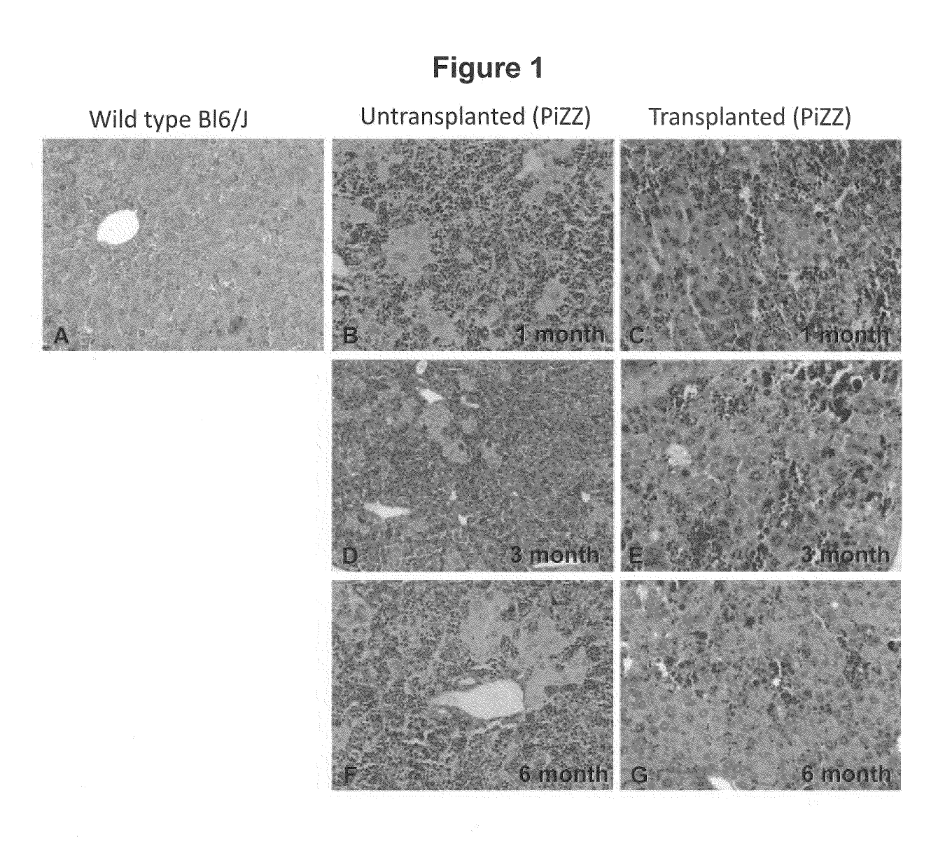

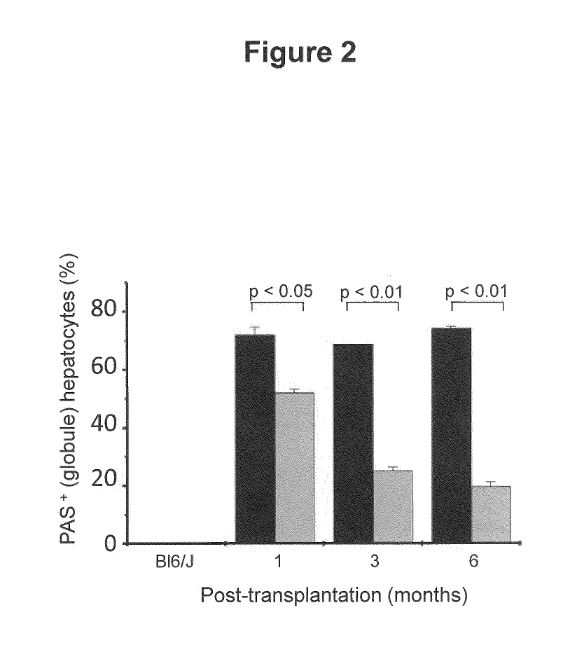

Presence of the diastase resistant globules in the hepatocytes of the PiZZ mice is the hallmark of A1-ATD. The 5 μm parafin sections were used to study the diastase resistant globules in the hepatocytes. The sections were deparaffinized and hydrated to deionized water. Sections were treated with 0.5% diastase in microwave at 600 watts for 25 seconds. The sections were then rinsed in running water and treated with periodic acid solution in microwave at 800 watts for 10 seconds. All slides were rinsed with water for several times and then treated with Schiff's reagent in microwave at 800 watts for another 15 seconds. Then the Schiff's reagent is mixed and incubated for 1 minute. All the slides were rinsed in gentle running warm water for 5 minutes. All the slides were placed in Hematoxylin containing coupler jar and micro-waved at 800 watts for 10 seconds and rinsed in tap water for 1-2 minutes. Slides were rapidly dehydrated and mounted with DPX (mounting medium). The percentages of diastase resistant globules in the hepatocytes were calculated by taking random images of 30 non-overlapping fields at 200× magnification. Each image was analyzed for the presence of globules by using ImageJ (Cell counter) software. (FIG. 1 and FIG. 2).

### Example 6

Analysis of the Percentage Collagen proportionate Area in the Transplanted PiZZ Mice

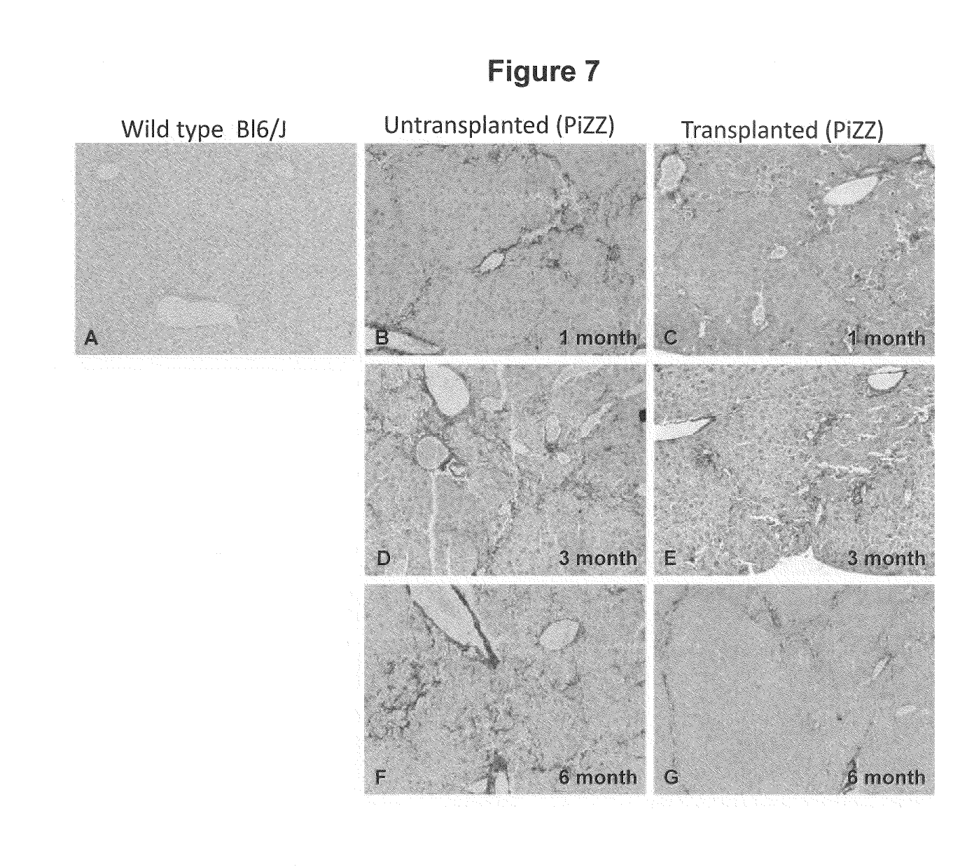

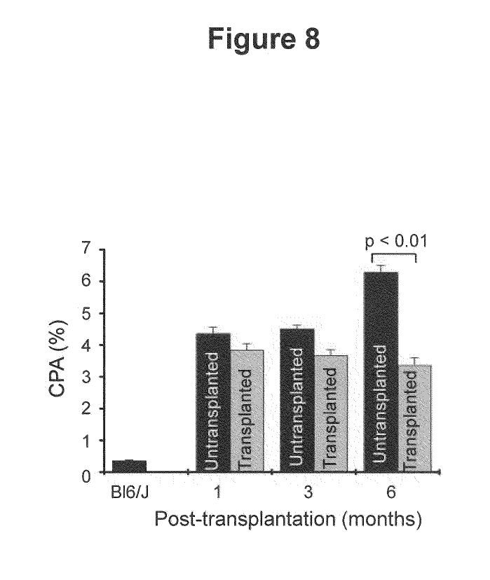

A1-ATD causes damage in the liver which can lead to fibrosis/cirrhosis and hepatocellular carcinoma. Different stages of fibrosis were studied as per standard protocols. Picrosirus red staining was done according to the manufacturer's protocol. Briefly, paraffin sections are rehydrated with distilled water and stained with the Hematoxylin for 10 min. The paraffin sections were rinsed well with distilled water. The sections were placed in solution A containing Phosphomolybdic acid hydrate for 2 minutes. They were then rinsed in distilled water and placed in solution B containing Direct Red 80 2,4,6-trimitrophenol for 110 minutes. Then all the slides were placed in solution C containing 0.01 N HCl solution for 2 minutes. All slides were dehydrated, cleared and mounted in DPX (mounting medium). Imaging was done using an Olympus microscope equipped with a digital camera. Percentage of collagen proportionate area (CPA) was calculated by taking random images of 30 non-overlapping fields in 100× magnification. Each image was analyzed for the collagen area by using ImageJ software. (FIG. 7 and FIG. 8).

### Example 7

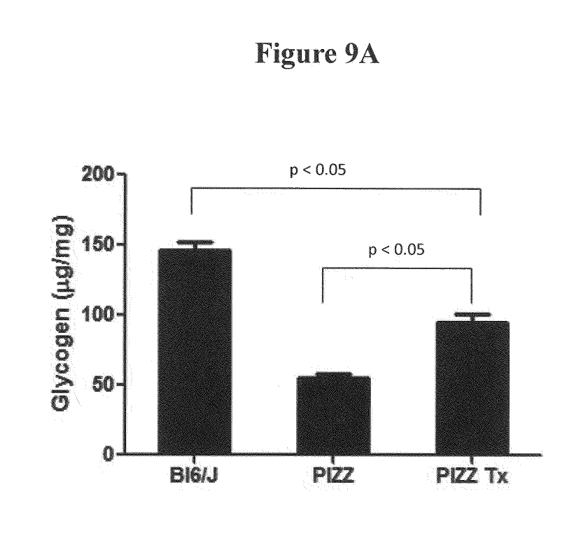

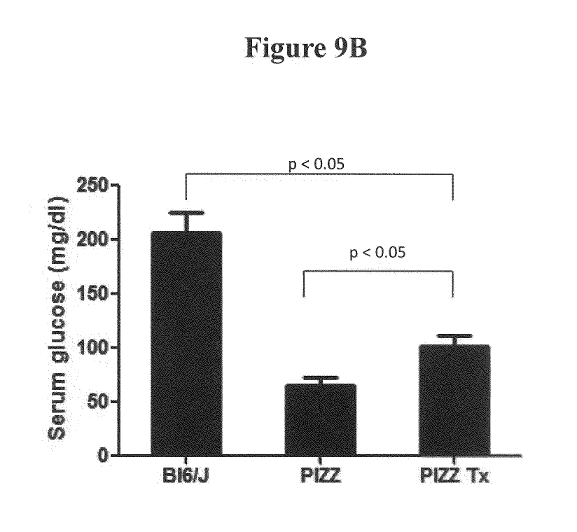

A1-ATD in transgenic C57B16/J mice expressing human mutant A1-AT (PiZZ), mimics the conformational abnormalities of the Z protein in the hepatocytes and induces autophagy. The autophagy, an intra cellular pathway that can apparently contribute to the degradation and recycling of unfolded proteins and that autophagy interferes with glycogen metabolism. Evaluation of the glycogen levels in liver tissue and glucose in serum was done in wild type, control PiZZ mice and Lin⁻ transplanted PiZZ mice by enzymatic method as described by the Roehrig and Alfred (1974). Briefly, the liver tissue samples were frozen quickly in liquid nitrogen and stored at −70° C. until further assessment. Glycogen estimation is based on the incubation of liver homogenate with amyloglycosidase, which degrades the glycogen to glucose. The glucose obtained from the liver tissue and in the serum samples were determined by GOD-POD method according to protocol provided by manufacturer of the kit (ERBA Mannheim-Transasia, India). Absorbance of the samples were read on 505/670 nm by using KC junior software with an ELISA reader (Powerwave XS-Biotech). The results are shown in FIGS. 9A and 9B.

### Example 8

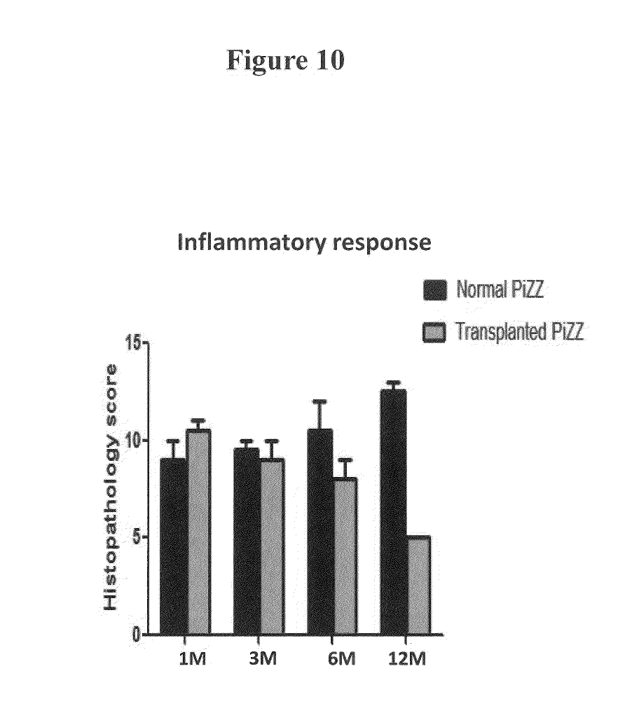

A1-ATD in transgenic mice expressing human mutant A1-AT (PiZZ), showed significant immune reaction as it has human mutated A1-AT, which causes death of hepatocytes. Inflammatory reactions in different anatomical locations of the control PiZZ mice and Lin⁻ transplanted PiZZ mice liver sections were scored by a standard protocol, (El-Hayek et al., 2005). Briefly, paraffin sections were stained with Hematoxylene and Eosin and the scoring was done on the basis of portal inflammation, bile duct inflammation, periportal necro-inflammatory activity, lobular inflammatory activity, confluent necrosis, endotheliasis and sinusoidal lymphocyte inflammation (FIG. 10).

References cited

| | | | |
|---|---|---|---|
| U.S. Pat. No. 6,403,646 | B1 | June, 2002 | Perlmutter et al. |
| U.S. Pat. No. 6,656,912 | B2 | December, 2003 | Perlmutter et al. |

De Serres F J. Worldwide racial ethnic distribution of A1-AT deficiency: Summary of an analysis of published genetic, epidemiologic surveys. Chest 122:2002, 1817-1829.

Ding J, Yannan G R, Roy-Chowdhury N, Hidvegi T, Basma H, Rennard S I, et al. (2011). Spontaneous hepatic repopulation in transgentic mice expressing mutant human α1-antitrypsin by wild-type donor hepatocytes. J Clin Invest 121, 1930-1934.

Lomas D A, Evans D L, Finch J AT, Carrell R W, 1992. The mechanism of Z α1-antitrypsin accumulation in the liver, Nature, 357(6379):605-607.

Carlson J A, Rogers B B, Sifers R N, Finegold M J, Clift S M, DeMayo F J, Bullock D W, Woo S L. 1989. Accumulation of PiZ antitrypsin causes liver damage in transgenic mice. J. Clin. Invent. 83:1183-90.

Dycaico M J, Grant S C, Felts K, Nichols W S, Geller S A, Hager J H, Pollard A J, Kohler S W, Short H P, Jirik F R, Hanahan D, Sorge J A. 1988. Neonatal hepatitis induced by α1-antitrypsin: A transgenic mouse model. Science. 242:1409-12.

Whitington P F., Alonso E M, Boyle J T, Molleston J P, Rosenthal P, Emond J C and Millis J M. 1998. Liver transplantation for the treatment of urea cycle disorders. J. Inh. Met. Dis. 21: 112-118.

Beckermann B M., Kallifatidis G., Groth A, Frommhold D., Apel A., Mattern J., Salnikov A V., Moldenhuer G., Wagner W., Diehlm V, VEGF expression by mesenchymal stem cell contributes to angiogenesis in pancreatic carcinoma. Br J Cancer. 2008 Aug. 19; 99(4): 622-631.

Kayler L K, Rasmussen C S, Dykstra D M, Punch J D, Ru-dich S M, Magee J C, Maraschio M A, Arenas J D, Campbell D. A Jr, Merion R M. 2003. Liver transplantation in children with metabolic disorders in the United States. Am. J. Transplant, 3: 334-339.

Fox, I. J., Chowdhury, J. R., Kaufman, S. S., Goertzen, T. C., Chowdhury, N. R., Warkentin, P. I., Dorko, K., Sauter, B. V., and Strom, S. C. 1998. Treatment of the Cliger-Najjar syndrome type 1 with hepatocytes transplantation. N. Engl. J. Med. 338: 1422-1426.

Strom, S. C., Rubinstein, W. S., Barranger, J. A., Towbin, R. B., Charon, M., Mieles, L., Pisarov, L. A., Dorko, K., Thompson, M. T., and Reyes, J. 1997. Transplantation of human hepatocytes. J. Trans. Proc. 29: 2103-2106.

Yadav N, Kanjirakkuzbiyil S, Kumar S, Jain M, Halder A, Saxena R, Mukhopadhyay A. The therapeutic effect of bone marrow derived liver cells in the phenotypic correction of murine hemophila A. Blood 2009:114: 4552-456.

Kim D W., Staples M., Shinozuka K., Pantcheva P., Kang S D., Borlongan C V. Wharton's Jelly-Derived mesenchymal Stem Cells: Phenotypic Characterization and Optimizing Their Therapuetic potential for Clinical Applications. Int. J. Mol. Sci. 2013, 14, 11692-11712.

Ghannam S, Bouffi C, Djouad F, Jorgensen C, Noël D. Immunosuppression by mesenchymal stem cells: mechanisms and clinical applications. Stem Cell Research & Therapy 2010, 1:2

Shi M, Liu Z W, Wang F S. Immunomodulatory properties and therapeutic application of mesenchymal stem cells. Clin Exp Immunol. 2011 April; 164(1): 1-8.

Follenzi A, Raut S, Merlin S, Sarkar R, Guptal S. Role of bone marrow transplantation for correcting hemophilia A in mice. Blood 2012; 119: 5532-5542.

Muraca m, Ferraresso C, Vilei M T, Gramato A, Quarta M, Cozzi E, et al. Liver repopulation with bone marrow derived cells improves the metabolic disorder in the Gunn rat. Gut 2207; S6:1725-1735.

Chen H L. Wang R, Chen H L., Hwu W L, Jeng Y M, Chang M H, et al. Bone marrow transplantation results in donor-derived hepatocytes in an animal model of inherited cholestatic liver disease. J Biomed Sci 2008; 15:615-22.

Laganse E, Connors H, Al-Dhalimy M, Reitsma M, Dohse M, Osborne L, Wange X, Finegold M, Weissman I L, Grompe M. Purified hematopoietic stem cells can differentiate into hepatocytes in vivo. Nat Med 2000; 6: 1229-1234

Stock P, Brückner S, Ebensing S, Hempel M, Dollinger M M and Christ B. 2010. The generation of hepatocytes from mesenchymal stem cells and engraftment into murine liver. Nature Protocols. 5(4): 617-627.

Rudnick D A, et al. Analysis of hepatoecellular proliferation in a mouse model of a1-AT deficiency. Hepatology 39, 2004, 1048-1055.

Roehrig K L, Allred J B. Direct enzymatic procedure for the determination of liver glycogen. Anal Biochem 1974;58:414-421.

Roy S, Javed S, Jain S K, Majundar S S, mukhopadhyay, A. Donor Hematopoietic Stem Cells Confer Long-Term Marrow Reconstitution by Self-Renewal Divisions Exceeding to That of Host Cells, PLoS ONE 2012,7:e50693.

El-Hayek, J. M., Rogers, T. E., and Brown, G. R. 2005. The role of TNF in hepatic histopathological manifestations and hepatic CD8+ T cell alloresponses in murine MHC class I disparate GVHD. J Leukoc Biol 78:1001-1007.

1. A method for treatment of A1-ATD comprising administering bone marrow-derived therapeutically active cells to a subject in need thereof.

2. The method of claim 1, wherein said cells comprise uncommitted progenitor cells that are purified from bone marrow.

3. The method of claim 1, wherein bone marrow-derived therapeutically active cells are BM-derived lin⁻ cells.

4. The method of claim 1, comprising administering about 0.25×10⁶ Lin⁻ cells per dose.

5. The method of claim 1, comprising administration of between about 0.1×10⁶ to about 20×10⁶ cells per dose per 100 gm of subject's liver.

6. The method of claim 1, wherein the cells are administered in an amount sufficient to reduce the number of misfolded A1-AT globules in the hepatic cytoplasm.

7. The method of claim 1, wherein transplantation of Lin⁻ BM cells causes significant increase in the glycogen level in the diseased liver tissue and glucose level in serum.

8. The method of claim 1, wherein transplantation leads to drop of inflammatory reaction.

9. The method of claim 1, wherein transplanted cells involves regeneration of hepatic parenchymal and non-parenchymal cells.

10. The method of claim 1, wherein the bone marrow-derived therapeutically active cells lead to replacement of hepatocytes expressing human mutant A1-AT with neo-hepatocyctes-derived from the BM cells.
